(19) United States
(12) Patent Application Publication
(10) Pub. No.: US 2021/0393231 A1
(43) Pub. Date: Dec. 23, 2021

(54) MEDICAL IMAGE DIAGNOSTIC SYSTEM, MEDICAL IMAGE DIAGNOSTIC METHOD, INPUT DEVICE, AND DISPLAY DEVICE

(71) Applicant: CANON MEDICAL SYSTEMS CORPORATION, Otawara-shi (JP)

(72) Inventor: Akira NISHIJIMA, Nasushiobara (JP)

(73) Assignee: CANON MEDICAL SYSTEMS CORPORATION, Otawara-shi (JP)

(21) Appl. No.: 17/350,678

(22) Filed: Jun. 17, 2021

(30) Foreign Application Priority Data
Jun. 19, 2020 (JP) 2020-105945

(51) Int. Cl. A61B 6/00; H04N 9/31; G16H 40/20; G16H 30/20; G16H 40/60; G16H 50/20; G06N 20/00

(52) U.S. Cl. CPC A61B 6/54; H04N 9/317; A61B 6/563; A61B 6/4458; A61B 6/032; G16H 30/20; G16H 40/60; G16H 50/20; G06N 20/00; G16H 40/20

(57) ABSTRACT

A medical image diagnostic system of an embodiment includes a processing circuit which is configured to control transition between a plurality of steps included in a workflow for examining a subject. The processing circuit is configured to acquire first information representing a preparation state of the subject in a first step among the plurality of steps, is configured to acquire second information representing permission for transition from the first step to a second step, and is configured to control transition from the first step to the second step on the basis of the first information and the second information.

### CROSS-REFERENCE TO RELATED APPLICATION

The present application claims priority based on Japanese Patent Application No. 2020-105945, filed on Jun. 19, 2020, the content of which is incorporated herein by reference.

### FIELD

Embodiments disclosed in the present description and drawings relate to a medical image diagnostic system, a medical image diagnostic method, an input device, and a display device.

### BACKGROUND

A shortage of doctors and engineers in the healthcare industry is becoming a serious problem. Meanwhile, with the advent of artificial intelligence and the improvement in data transmission speed and the amount of traffic able to be transmitted according to new wireless communication systems such as 5G and 6G, the demand for automatic diagnosis, remote diagnosis, and the like are increasing. If the price of and doses in X-ray computed tomography (CT) apparatuses will decrease in the future, X-ray CT apparatuses are expected to be more likely to be used for physical examination and the like. Since a contrast medium and a special scan technique are not necessary for physical examination, a procedure relating to examination is simple. However, even for such applications, there is a problem that examination may not be able to be frequently performed in provinces, developing countries, and the like due to a shortage of doctors and engineers. This problem is not limited to X-ray CT apparatuses and is common for other medical image capturing apparatuses (also referred to as medical image diagnostic apparatuses) such as magnetic resonance imaging (MRI) apparatuses, ultrasonic image diagnostic apparatuses, and nuclear medical diagnostic apparatuses.

### BRIEF DESCRIPTION OF THE DRAWINGS

FIG. 1 is a diagram showing a configuration example of a medial image diagnostic system in an embodiment.

FIG. 2 is a diagram showing a configuration example of a terminal device in an embodiment.

FIG. 3 is a diagram showing a configuration example of an X-ray CT apparatus in an embodiment.

FIG. 4 is a perspective view of a frame apparatus in an embodiment.

FIG. 5 is a flowchart showing an example of a flow of a series of processes of the X-ray CT apparatus in an embodiment.

FIG. 6 is a flowchart showing an example of a flow of a series of processes of the X-ray CT apparatus in the embodiment.

FIG. 7 is a diagram schematically showing a state in which a display is moved in accordance with the posture of a patient.

FIG. 8 is a diagram schematically showing a state in which the display is moved in accordance with the posture of the patient.

FIG. 9 is a flowchart showing a flow of a series of processes at the time of emergency stop of the X-ray CT apparatus in an embodiment.

FIG. 10 is a diagram showing an example of a projector in an embodiment.

FIG. 11 is a diagram showing a focal position adjustment method.

FIG. 12 is a diagram showing another configuration example of the terminal device in an embodiment.

### DETAILED DESCRIPTION

An object of embodiments disclosed in the present description and drawings is to examine a subject with safety and without impairing convenience. However, the object of the embodiments disclosed in the present description and drawings is not limited to the aforementioned object. Objects corresponding to the effects according to configurations described in embodiments which will be described later can also be assessed as other objects.

A medical image diagnostic system of an embodiment includes a processing circuit which is configured to control transition between a plurality of steps included in a workflow for examining a subject. The processing circuit is configured to acquire first information representing a preparation state of the subject in a first step among the plurality of steps, is configured to acquire second information representing permission for transition from the first step to a second step, and is configured to control transition from the first step to the second step on the basis of the first information and the second information.

Hereinafter, a medical image diagnostic system, a medical image diagnostic method, an input device, and a display device of embodiments will be described with reference to the drawings.

### [Configuration of Medical Image Diagnostic System]

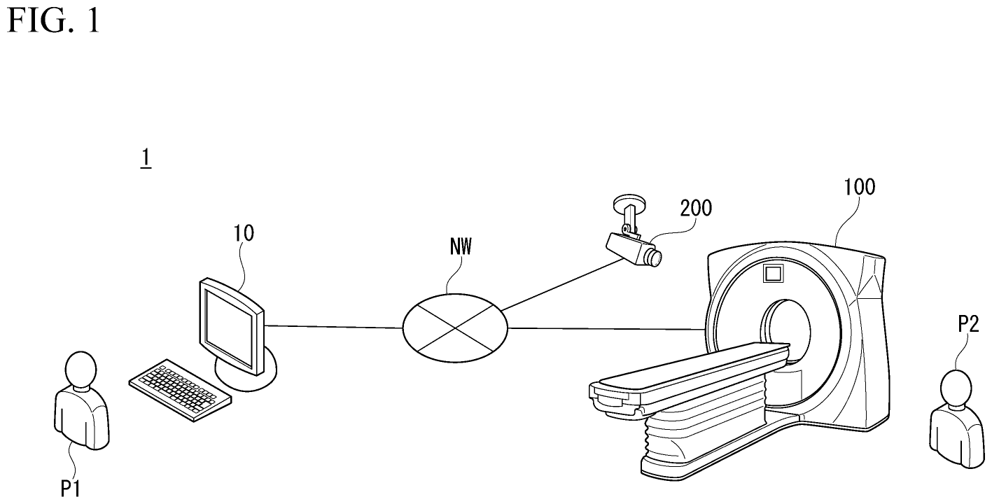

FIG. 1 is a diagram showing a configuration example of a medical image diagnostic system 1 in an embodiment. The medical image diagnostic system 1 includes, for example, a terminal device 10, a medical image capturing apparatus 100, and a camera 200. The terminal device 10, the medical image capturing apparatus 100, and the camera 200 are connected such that they can communicate through a communication network NW.

The communication network NW means a general information communication network using a telecommunication technology. The communication network NW includes a telephone communication line network, an optical fiber communication network, a cable communication network, a satellite communication network, and the like in addition to a wireless/wired LAN such as a hospital based local area network (LAN) and the Internet.

The terminal device 10 is a terminal device such as a personal computer, a tablet terminal, or a cellular phone used by a medical personnel member P1. The medical personnel member P1 is, for example, a medical worker such as a doctor, an engineer, or a nurse. For example, the medical personnel member P1 remotely operates the medical image capturing apparatus 100 or instructs a patient P2 who is a subject (subject person) using the terminal device 10.

The medical image capturing apparatus 100 is an apparatus that generates a medical image by scanning the patient P2 and allows diagnosis on the patient P2 on the basis of the medical image. For example, the medical image capturing apparatus 100 may be an X-ray CT apparatus, an MRI apparatus, an ultrasonic image diagnostic apparatus, a nuclear medical diagnostic apparatus, or the like. Hereinafter, an example in which the medical image capturing apparatus 100 is an X-ray CT apparatus will be described.

The camera 200 is attached to, for example, a ceiling, a wall, or the like of a CT room in which the X-ray CT apparatus 100 is installed. For example, the camera 200 images the patient P2 in the CT room and transmits an image in the CT room to the terminal device 10 through the communication network NW or the X-ray CT apparatus 100. An image of the camera 200 may be a still image or a moving image. The camera 200 may directly transmit a captured image to the terminal device 10 or indirectly transmit the captured image to the terminal device 10 through the X-ray CT apparatus 100. The camera 200 is an example of a “sensor.”

### [Configuration of Terminal Device]

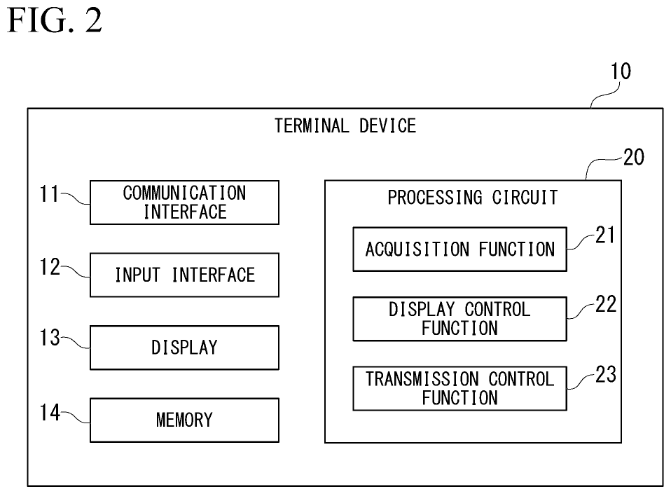

FIG. 2 is a diagram showing a configuration example of the terminal device 10 in an embodiment. The terminal device 10 includes, for example, a communication interface 11, an input interface 12, a display 13, a memory 14, and a processing circuit 20.

The communication interface 11 communicates with external apparatuses such as the X-ray CT apparatus 100 and the camera 200 through the communication network NW. The communication interface 11 includes, for example, a network interface card (NIC) or the like.

The input interface 12 receives various input operations from an operator (e.g., the medical personnel member P1), converts the received input operations into electrical signals, and outputs the electrical signals to the processing circuit 20. For example, the input interface 12 includes a mouse, a keyboard, a trackball, a switch, a button, a joystick, a touch panel, and the like. The input interface 12 may be, for example, a user interface that receives audio input, such as a microphone. When the input interface 12 is a touch panel, the input interface 12 may also include a display function of the display 13.

The input interface 12 in the present description is not limited to a component including physical operating parts such as a mouse and a keyboard. For example, an electrical signal processing circuit that receives an electrical signal corresponding to an input operation from an external input device provided separately from the device and outputs the electrical signal to a control circuit is also included in examples of the input interface 12.

The display 13 displays various types of information. For example, the display 13 displays an image generated by the processing circuit 20, a graphical user interface (GUI) for receiving various input operations from the medical personnel member P1, and the like. For example, the display 13 is a liquid crystal display (LCD), a cathode ray tube (CRT) display, an organic electroluminescence (EL) display, or the like.

The memory 14 is realized by, for example, a semiconductor memory element such as a random access memory (RAM) or a flash memory, a hard disk, or an optical disc. These non-transitory storage media may be realized by other storage devices connected through the communication network NW, such as a network attached storage (NAS) and an external storage server device. The memory 14 may include a non-transitory storage medium such as a read only memory (ROM) or a register.

The processing circuit 20 includes, for example, an acquisition function 21, a display control function 22, and a transmission control function 23. The processing circuit 20 realizes these functions, for example, by a hardware processor (computer) executing a program stored in the memory 14 (storage circuit).

The hardware processor means, for example, a circuitry such as a central processing unit (CPU), a graphics processing unit (GPU), an application specific integrated circuit (ASIC), or a programmable logic device (e.g., simple programmable logic device (SPLD), a complex programmable logic device (CPLD), or a field programmable gate array (FPGA)). The hardware processor may be configured such that the program is directly incorporated into the circuit of the hardware processor instead of being stored in the memory 14. In this case, the hardware processor realizes the functions by reading and executing the program incorporated into the circuit thereof. The aforementioned program may be stored in the memory 14 in advance or stored in a non-temporary storage medium such as a DVD or a CD-ROM and installed to the memory 14 from the non-temporary storage medium when the non-temporary storage medium is inserted into a drive device (not shown) of the terminal device 10. The hardware processor is not limited to a configuration as a single circuit and may be configured as a single hardware processor by combining a plurality of independent circuits to realize each function. A plurality of components may be integrated into a single hardware processor to realize each function.

The acquisition function 21 acquires an image of the CT room from the camera 200 through the communication interface 11 and acquires control information and vital information from the X-ray CT apparatus 100 through the communication interface 11. The control information is various types of information for controlling the X-ray CT apparatus 100 to scan the patient P2. The vital information is, for example, numerical value information about vital signs such as a heart rate, a pulse rate, a blood pressure, a respiration rate, and a body temperature. The acquisition function 21 may acquire a medical image (hereinafter referred to as a CT image) obtained through X-ray imaging (scanning) of the X-ray CT apparatus 100 from the X-ray CT apparatus 100 through the communication interface 11. A CT image may be a single tomographic image or a plurality of tomographic images. A CT image may be a plurality of time phase images or a captured image.

The display control function 22 causes the display 13 to display an image of the CT room, control information, vital information, a CT image, and the like acquired by the acquisition function 21.

The transmission control function 23 transmits information input to the input interface 12 to the X-ray CT apparatus 100 through the communication interface 11.

### [Configuration of X-Ray CT Apparatus]

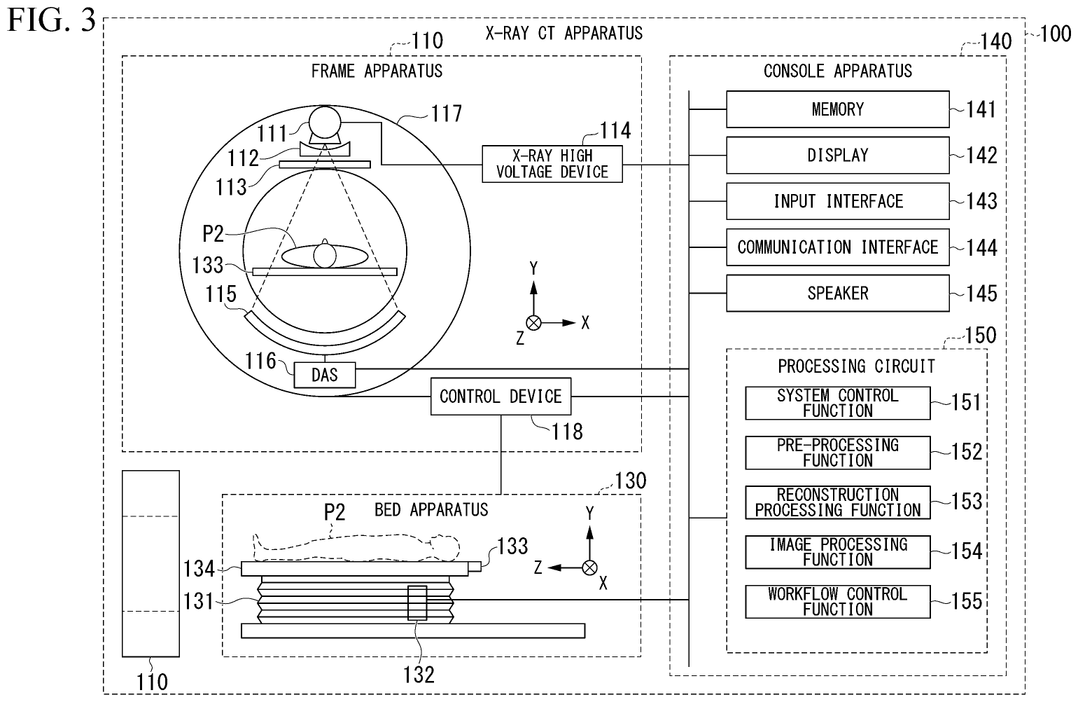

FIG. 3 is a diagram showing a configuration example of the X-ray CT apparatus 100 in an embodiment. The X-ray CT apparatus 100 includes, for example, a frame apparatus 110, a bed apparatus 130, and a console apparatus 140. Although FIG. 3 shows a figure in which the frame apparatus 110 is viewed in a Z-axis direction and a figure in which the frame apparatus 110 is viewed in an X-axis direction for convenience of description, there is one frame apparatus 100 in practice. In an embodiment, a rotation axis of a rotary frame 117 in a non-tilted state or a longitudinal direction of a top board 133 of the bed apparatus 130 is defined as the Z-axis direction, an axis perpendicular to the Z-axis direction and parallel to the floor is defined as the X-axis direction, and a direction orthogonal to the Z-axis direction and perpendicular to the floor is defined as a Y-axis direction.

The frame apparatus 110 includes, for example, an X-ray tube 111, a wedge 112, a collimator 113, an X-ray high voltage device 114, an X-ray detector 115, a data collecting system (hereinafter, data acquisition system (DAS)) 116, the rotary frame 117, and a control device 118.

The X-ray tube 111 generates X-rays by applying a high voltage from the X-ray high voltage device 114 or radiating thermoelectrons from a cathode (filament) to an anode (target). The X-ray tube 111 includes a vacuum tube. For example, the X-ray tube 111 is a rotating anode X-ray tube that generates X-rays by radiating thermoelectrons to a rotating anode.

The wedge 112 is a filter for controlling an X-ray dose radiated from the X-ray tube 111 to the patient P2. The wedge 112 attenuates X-rays being transmitted through the wedge 112 such that a distribution of the X-ray dose radiated from the X-ray tube 111 to the patient P2 becomes a predetermined distribution. The wedge 112 is also called a wedge filter or a bow-tie filter. The wedge 112 is obtained, for example, by processing aluminum to have a predetermined target angle and a predetermined thickness.

The collimator 113 is a mechanism for narrowing a radiation range of X-rays that have been transmitted through the wedge 112. The collimator 113 narrows the radiation range of X-rays, for example, by forming a slit using a combination of a plurality of lead plates. The collimator 113 may be called an X-ray diaphragm.

The X-ray high voltage device 114 includes, for example, a high voltage generation device and an X-ray control device. The high voltage generation device has an electric circuit including a transformer (trans), a rectifier, and the like and generates a high voltage to be applied to the X-ray tube 111. The X-ray control device controls an output voltage of the high voltage generation device in response to an X-ray dose that needs to be generated by the X-ray tube 111. The high voltage generation device may boost a voltage through the aforementioned transformer or an inverter. The X-ray high voltage device 114 may be provided in the rotary frame 117 or provided on the side of a fixed frame (not shown) of the frame apparatus 110.

The X-ray detector 115 detects the intensity of X-rays that have been generated by the X-ray tube 111, have passed through the patient P2 and applied thereto. The X-ray detector 115 outputs an electrical signal (or an optical signal or the like) in response to the detected intensity of X-rays to the DAS 116. The X-ray detector 115 includes, for example, a plurality of X-ray detection element columns. The plurality of X-ray detection element columns are a plurality of X-ray detection elements arranged in a channel direction along an arc having a focal point of the X-ray tube 111 as a center. The plurality of X-ray detection element columns are arranged in a slice direction (column direction, a row direction).

The X-ray detector 115 is an indirect detector including a grid, a scintillator array, and an optical sensor array, for example. The scintillator array includes a plurality of scintillators. Each scintillator includes scintillator crystals. The scintillator crystals emit light in a quantity of light in response to the intensity of incident X-rays. The grid includes an X-ray shielding plate that is disposed on a side of the scintillator array on which X-rays are incident and has a function of absorbing scattering X-rays. The grid may also be called a collimator (one-dimensional collimator or two-dimensional collimator). The optical sensor array includes, for example, optical sensors such as photomultiplier tubes (photomultipliers (PMT)). The optical sensor array outputs an electrical signal in response to the quantity of light emitted from the scintillators. The X-ray detector 115 may be a direct conversion type detector having a semiconductor element that converts incident X-rays into an electrical signal.

The DAS 116 includes, for example, an amplifier, an integrator, and an A/D converter. The amplifier performs amplification processing on an electrical signal output from each X-ray detection element of the X-ray detector 115. The integrator integrates the amplified electrical signal over a view period (which will be described later). The A/D converter converts an electrical signal representing an integration result into a digital signal. The DAS 116 outputs detection data based on a digital signal to the console apparatus 140. Detection data is digital values of a channel number and a column number of an X-ray detection element that is a generation source, and an X-ray intensity identified by a view number representing a collected view. A view number is a number that changes according to rotation of the rotary frame 117, for example, a number increasing according to rotation of the rotary frame 117. Accordingly, a view number is information representing a rotation angle of the X-ray tube 111. A view period is a period from a rotation angle corresponding to a certain view number until a rotation angle corresponding to the next view number. The DAS 116 may detect switching between views according to a timing signal input from the control device 118, an internal timer, or a signal acquired from a sensor that is not illustrated. When X-rays are continuously exposed by the X-ray tube 111 in the case of full scanning, the DAS 116 collects detection data groups of the entire circumference (360 degrees). When X-rays are continuously exposed by the X-ray tube 111 in the case of half scanning, the DAS 116 collects detection data of half a circumference (180 degrees).

The rotary frame 117 is an annular rotary member that rotates the X-ray tube 111, the wedge 112, the collimator 113, and the X-ray detector 115 in a state in which they are held with the X-ray tube 111, the wedge 112 and the collimator 113 facing the X-ray detector 115. The rotary frame 117 is rotatably supported by a fixed frame with the patient P2 introduced thereinto as a center. The rotary frame 117 further supports the DAS 116. Detection data output from the DAS 116 is transmitted from a transmitter including a light-emitting diode (LED) provided in the rotary frame 117 to a receiver including a photodiode provided in a non-rotary part (e.g., the fixed frame) of the frame apparatus 110 through optical communication and forwarded to the console apparatus 140 through the receiver. A method of transmitting detection data from the rotary frame 117 to the non-rotary part is not limited to the aforementioned method using optical communication, and an arbitrary contactless transmission method may be employed. The rotary frame 117 is not limited to an annular member and may be a member such as an arm which can support and rotate the X-ray tube 111 and the like.

The control device 118 includes, for example, a processing circuit having a processor such as a CPU and a driving mechanism including a motor, an actuator, and the like. The control device 118 receives an input signal from an input interface 143 provided in the console apparatus 140 or the frame apparatus 110 and controls operations of the frame apparatus 110 and the bed apparatus 130.

For example, the control device 118 rotates the rotary frame 117, tilts the frame apparatus 110, and moves the top board 133 of the bed apparatus 130. When tilting the frame apparatus 110, the control device 118 rotates the rotary frame 117 on an axis parallel to the Z-axis direction on the basis of an inclination angle (tilt angle) input to the input interface 143. The control device 118 ascertains a rotation angle of the rotary frame 117 according to an output of a sensor that is not illustrated, and the like. The control device 118 provides the rotation angle of the rotary frame 117 to a processing circuit 150 at any time. The control device 118 may be provided in the frame apparatus 110 or may be provided in the console apparatus 140.

The control device 118 causes the frame apparatus 110 to move along a moving rail to perform main scan imaging or perform scan imaging that is positioning imaging performed before execution of main scan imaging.

The bed apparatus 130 is an apparatus that introduces the patient P2 that is a scanning target placed thereon into the rotary frame 117 of the frame apparatus 110. The bed apparatus 130 includes, for example, a base 131, a bed driving device 132, the top board 133, and a support frame 134. The base 131 includes a housing that supports the support frame 134 such that the support frame 134 can move in the vertical direction (Y-axis direction). The bed driving device 132 includes a motor and an actuator. The bed driving device 132 moves the top board 133 on which the patient P2 is placed in the longitudinal direction (Z-axis direction) of the top board 133 along the support frame 134. The top board 133 is a board-shaped member on which the patient P2 is placed.

The console apparatus 140 includes, for example, a memory 141, a display 142, the input interface 143, a communication interface 144, a speaker 145, and the processing circuit 150. Although the console apparatus 140 is described as a body separate from the frame apparatus 110 in the present embodiment, some or all components of the console apparatus 140 may be included in the frame apparatus 110.

The memory 141 is realized by, for example, a semiconductor memory element such as a RAM or a flash memory, a hard disk, an optical disk, or the like. The memory 141 stores, for example, detection data, projection data, reconstructed images, CT images, and the like. These types of data may be stored in an external memory with which the X-ray CT apparatus 100 can communicate instead of the memory 141 (or in addition to the memory 141). The external memory is controlled, for example, by a cloud server that manages the external memory and receives read/write requests. The memory 141 stores a scan workflow. The scan workflow is pattern information in which a series of steps (processing procedure) for controlling the X-ray CT apparatus 100 has been determined. The scan workflow may be replaced with a program, a program component, an algorithm, a sequence, or the like.

The display 142 displays various types of information. For example, the display 142 displays CT images generated by the processing circuit 150, GUI images through which various operations are received from an operator (e.g., patient P2), and the like. The display 142 is, for example, a liquid crystal display, a CRT, an organic EL display, or the like. The display 142 may be provided in the frame apparatus 110. The display 142 may be a desktop type or a display device (e.g., a tablet terminal) capable of wirelessly communicating with the main body of the console apparatus 140.

The input interface 143 receives various input operations of the operator (e.g., patient P2) and outputs electrical signals representing details of the received input operations to the processing circuit 150. For example, the input interface 143 receives input operations such as collection conditions when detection data or projection data (which will be described later) is collected, reconstruction conditions when a CT image is reconstructed, and image processing conditions when a post-processing image is generated from a CT image. For example, the input interface 143 is realized by a mouse, a keyboard, a touch panel, a trackball, a switch, a button, a joystick, a foot pedal, a camera, an infrared sensor, a microphone, or the like. The input interface 143 may be provided in the frame apparatus 110. The input interface 143 may be realized by a display device (e.g., a table terminal) capable of wirelessly communicating with the main body of the console apparatus 140. The input interface 143 in the present description is not limited to a component including a physical operating part such as a mouse or a keyboard. For example, an electrical signal processing circuit that receives an electrical signal corresponding to an input operation from an external input device provided separately from the apparatus and outputs the electrical signal to a control circuit is also included in examples of the input interface 143.

The communication interface 144 includes, for example, an NIC, a wireless communication module, or the like. The communication interface 144 communicates with external devices such as the terminal device 10 and the camera 200 through the communication network NW.

The speaker 145 is disposed at a position at which the operator (e.g., patient P2) can hear sound. The speaker 145 outputs sound on the basis of information output from the processing circuit 150.

The processing circuit 150 controls the overall operation of the X-ray CT apparatus 100. The processing circuit 150 executes, for example, a system control function 151, a pre-processing function 152, a reconstruction processing function 153, an image processing function 154, a workflow control function 155, and the like. The processing circuit 150 realizes, for example, these functions by a hardware processor executing various programs such as a scan workflow stored in the memory 141.

The hardware processor means, for example, a circuitry such as a CPU, a GPU, an application specific integrated circuit, or a programmable logic device (e.g., simple programmable logic device, a complex programmable logic device, or a field programmable gate array). The hardware processor may be configured such that programs are directly incorporated into the circuit of the hardware processor instead of being stored in the memory 141. In this case, the hardware processor realizes the functions by reading and executing the programs incorporated into the circuit thereof. The hardware processor is not limited to a configuration as a single circuit and may be configured as a single hardware processor by combining a plurality of independent circuits to realize each function. A plurality of components may be integrated into a single hardware processor to realize each function.

The components included in the console apparatus 140 or the processing circuit 150 may be distributed and realized by a plurality of hardware components. The processing circuit 150 may be realized by a processing device capable of communicating with the console apparatus 140 instead of being included in the console apparatus 140. For example, the processing device is a workstation connected to a single X-ray CT apparatus or a device (e.g., a cloud server) that is connected to a plurality of X-ray CT apparatuses and collectively executes the same processes as those of the processing circuit 150.

The system control function 151 controls various functions of the processing circuit 150 on the basis of input operations received through the input interface 143.

The pre-processing function 152 performs pre-processing such as logarithmic conversion processing, offset correction processing, inter-channel sensitivity correction processing, and beam hardening correction processing on detection data output from the DAS 116 to generate projection data and stores the generated projection data in the memory 141.

The reconstruction processing function 153 performs reconstruction processing using a filtered back projection method, a successive approximation reconstruction method, or the like on the projection data generated by the pre-processing function 152 to generate a CT image and stores the generated CT image in the memory 141.

The image processing function 154 converts the CT image into a three-dimensional image or cross-section image data of an arbitrary cross section through a known method on the basis of an input operation received through the input interface 143. Conversion into the three-dimensional image may be performed by the pre-processing function 152.

The workflow control function 155 controls detection data collection processing in the frame apparatus 110 by controlling the X-ray high voltage device 114, the DAS 116, the control device 118, and the bed driving device 132 according to a scan workflow stored in the memory 141. The workflow control function 155 controls operations of functions when imaging for collecting scan images and imaging for collecting CT images used for diagnosis are performed according to the scan workflow stored in the memory 141.

The workflow control function 155 induces the patient P2 to mount on the top board 133 of the bed apparatus 130, induces the patient P2 to take a posture and an action (e.g., raising both hands and holding the breath, and the like) suitable to a scanning part, induces the patient P2 to dismount from the top board 133 of the bed apparatus 130, and confirms the intention of the patient P2 at a timing, such as before scanning, during scanning, or after scanning, by controlling the display 142, the input interface 143, the communication interface 144, and the speaker 145 according to the scan workflow stored in the memory 141. That is, the workflow control function 155 performs various types of processing for CT scanning while having a conversation with the patient P2 according to the scan workflow such that the patient P2 can perform scanning by himself/herself using the X-ray CT apparatus 100 even when the medical personnel member P1 is not around the patient P2 (interactively performs processing).

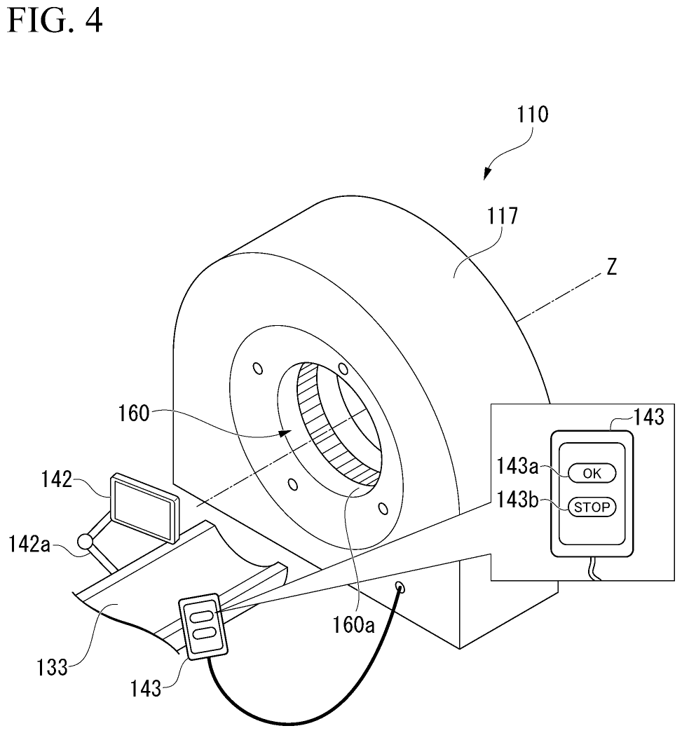

FIG. 4 is a perspective view of the frame apparatus 110 in an embodiment. As illustrated, an approximately cylindrical opening 160 is formed in the housing of the frame apparatus 110. The top board 133 of the bed apparatus 130 on which the patient P2 is placed is inserted into the opening 160. The above-described X-ray tube 111, the wedge 112, the collimator 113, the X-ray high voltage device 114, the X-ray detector 115, the DAS 116, the rotary frame 117, the control device 118, and the like are included in the housing of the frame apparatus 110.

The input interface 143 is attached to the housing of the frame apparatus 110 through a cable, for example. The input interface 143 is connected to the control device 118 of the frame apparatus 110 and the processing circuit 150 of the console apparatus 140 using a wire and transmits/receives data. The length of the cable connecting the frame apparatus 110 and the input interface 143 may be appropriately determined to a degree that can be operated by the patient P2 while lying down on the top board 133 of the bed apparatus 130. The input interface 143 may be connected to the control device 118 of the frame apparatus 110 and the processing circuit 150 of the console apparatus 140 wirelessly instead of using a wire such as a cable. In this case, the input interface 143 may be a wearable device that can be put on a wrist or the like of the patient P2.

The input interface 143 includes a first button 143a (“OK button” in the figure) by which the patient P2 consents progress to the next step of the scan workflow and a second button 143b (“STOP button” in the figure) by which the patient P2 does not consent progress to the next step of the scan workflow and stops processing of the current step. The first button 143a and the second button 143b may be physical (or tangible) buttons or virtual (or non-tangible) buttons. For example, when the input interface 143 is a touch panel, the first button 143a and the second button 143b may be virtual buttons.

When the input interface 143 is a wearable device, the first button 143a and the second button 143b may not be necessarily provided. For example, when the input interface 143 that is a wearable device is put on a wrist of the patient P2, the input interface 143 may recognize whether the patient P2 has consented progress to the next step or has requested stop of processing of the current step without consenting according to a hand motion of the patient P2, such as opening his/her palm or clenching his/her fist. That is, the input interface 143 may recognize an input operation according to a gesture of the patient P2.

The display 142 is attached to the top board 133 of the bed apparatus 130, for example, through a robot arm 142a. For example, the workflow control function 155 moves the robot arm 142a by driving an actuator that is not illustrated to move the screen of the display 142 to the line of sight of the patient P2. Accordingly, the patient P2 is caused to recognize various images.

For example, the workflow control function 155 may control a projector 190 capable of projecting images to a wall surface 160a of the opening 160 of the frame apparatus 110, the ceiling of the CT room, or the like instead of controlling the robot arm 142a to which the display 142 is attached.

### [Overall Flow of X-Ray CT Apparatus]

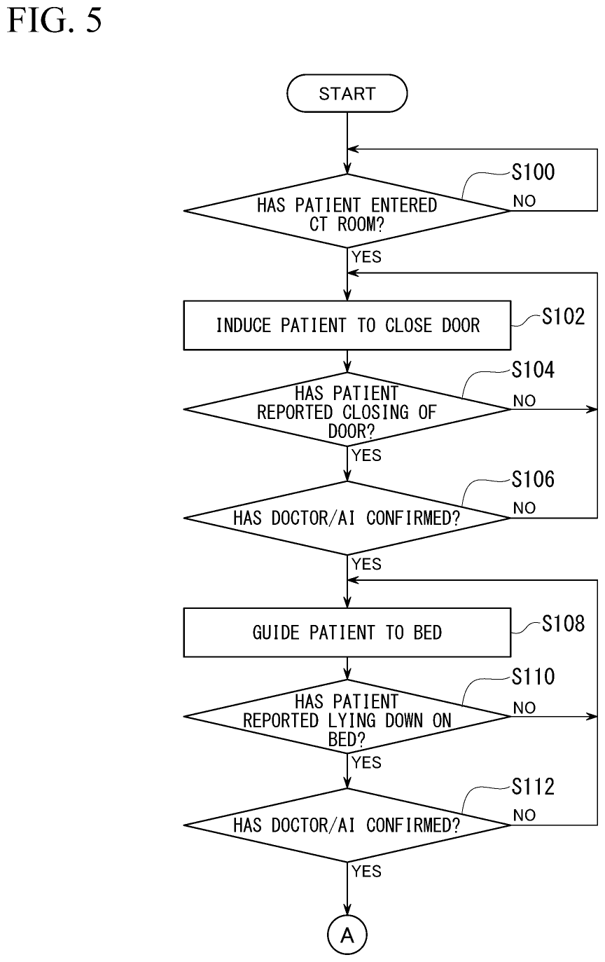

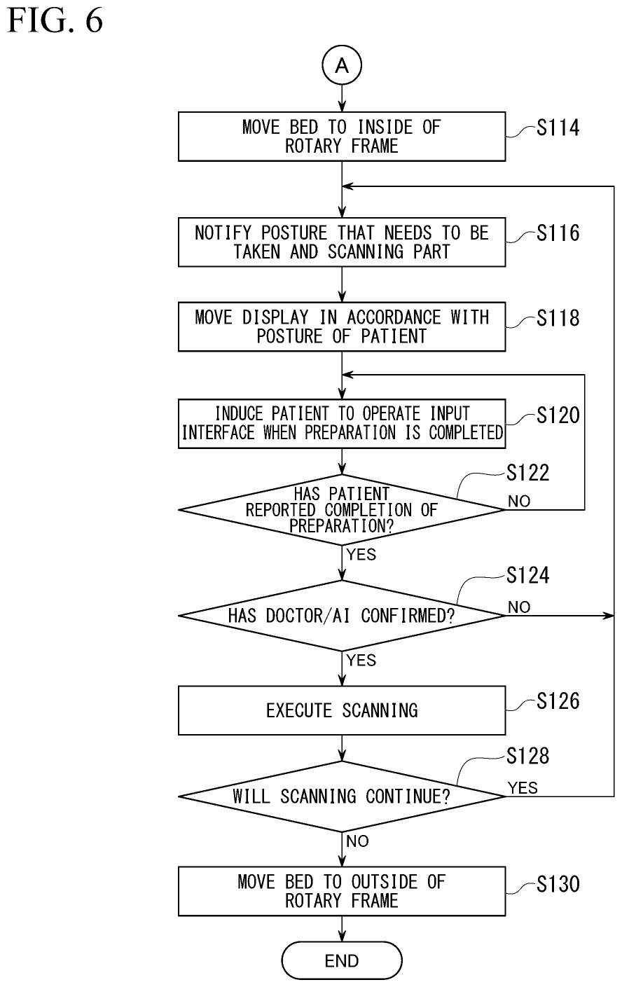

An example of processing of the X-ray CT apparatus 100 configured as above will be described below. FIG. 5 and FIG. 6 are flowcharts showing an example of a flow of a series of processes of the X-ray CT apparatus 100 in an embodiment. First, the workflow control function 155 determines whether the patient P2 has entered the CT room (step S100).

For example, the workflow control function 155 may acquire an image (a still image or a moving image) of the inside of the CT room from the camera 200 through the communication interface 144 and determine whether the patient P2 has entered the CT room on the basis of the acquired image. For example, there are cases in which an electric door that automatically or semi-automatically opens and closes is provided in the CT room and a sensor that detects opening/closing is provided in the electric door. In this case, the workflow control function 155 may acquire an electrical signal with respect to opening/closing of the door from the sensor through the communication interface 144 and determine whether the patient P2 has entered the CT room on the basis of the acquired signal.

The workflow control function 155 induces the patient P2 to close the door of the CT room upon determining that the patient P2 has entered the CT room (step S102).

For example, the workflow control function 155 causes the display 142 to display characters or an image for inducing the patient P2 to close the door of the CT room or causes the speaker 145 to output voice for inducing the patient P2 to close the door of the CT room. Accordingly, it is possible to suppress radiation leakage from the CT room and reduce a leaking radiation dose of the CT room.

Next, the workflow control function 155 determines whether the patient P2 has reported closing of the door (step S104).

For example, it is assumed that the patient P2 is induced to operate the first button 143a of the input interface 143 when the door is closed and the first button 143a of the input interface 143 has been operated by the patient P2 as a result. In this case, the input interface 143 outputs a signal representing that the first button 143a has been operated to the processing circuit 150. The signal representing that the first button 143a has been operated is an example of “first information.”

When the signal representing that the first button 143a has been operated cannot be acquired from the input interface 143, the workflow control function 155 determines that the patient P2 has not reported closing of the door. In this case, the workflow control function 155 returns to the process of S102 and continues to induce the patient P2 to close the door of the CT room.

On the other hand, when the signal representing that the first button 143a has been operated is acquired from the input interface 143, the workflow control function 155 determines that the patient P2 has reported closing of the door. Then, the workflow control function 155 determines whether the remotely located medical personnel member P1 has confirmed that the patient P2 has closed the door (step S106).

As described above, the image of the camera 200 is transmitted to the terminal device 10 and the image of the CT room is displayed on the display 13 of the terminal device 10. For example, when the medical personnel member P1 has confirmed that the patient P2 has closed the door by viewing the image of the CT room displayed on the display 13, the medical personnel member P1 inputs a confirmation result representing closing of the door to the input interface 12 of the terminal device 10. In other words, when the medical personnel member P1 has confirmed that the patient P2 has closed the door, the medical personnel member P1 inputs information for permitting transition to the next step of the scan workflow to the input interface 12 of the terminal device 10. Upon receiving this, the transmission control function 23 of the terminal device 10 transmits information indicating the confirmation result representing closing of the door (permission for transition to the next step) to the X-ray CT apparatus 100 through the communication interface 11. The workflow control function 155 determines that the remotely located medical personnel member P1 has confirmed that the patient P2 has closed the door when the communication interface 144 receives the information indicating the aforementioned confirmation result from the terminal device 10.

The workflow control function 155 may determine whether the patient P2 has closed the door using artificial intelligence (AI) as the process of S106. For example, the workflow control function 155 determines whether the patient P2 has closed the door by inputting the image (i.e., the image of the inside of the CT room) of the camera 200 to a machine learning model (hereinafter, an opening/closing determination model) MDL1 trained in advance to determine opening/closing of the door. The opening/closing determination model MDL1 is an example of a “trained model.”

The opening/closing determination model MDL1 is, for example, a model implemented by a neural network such as a convolutional neural network (CNN). The opening/closing determination model MDL1 is a model supervised-trained on the basis of training data in which correct answer information representing opening/closing states of the door of the CT room is associated as labels (also called targets) with images of the inside of the CT room. This correct answer information may be, for example, a two-dimensional vector having a probability al representing that the door is open and a probability a2 representing that the door is closed as elements. The training data may be replaced with data sets obtained by combining input data and output data when images of the inside of the CT room are the input data and the correct answer information representing opening/closing states of the door of the CT room is the output data. When an image of the CT room is input, the opening/closing determination model MDL1 outputs information representing whether the door of the CT room is open or closed by learning the opening/closing determination model MDL1 using such training data.

For example, the workflow control function 155 may determine that the patient P2 has closed the door when the opening/closing determination model MDL1 to which the image of the camera 200 has been input outputs a vector in which the probability a2 representing that the door is closed is higher than the probability al representing that the door is open (α2>α1) and determine that the patient P2 has not closed the door when the opening/closing determination model MDL1 outputs a vector in which the probability α1 representing that the door is open is higher than the probability α2 representing that the door is closed (α1>α2).

The training data for learning the opening/closing determination model MDL1 may be data sets in which the correct answer information representing opening/closing states of the door of the CT room and control information of the X-ray CT apparatus 100 are associated as labels with images of the inside of the CT room. The control information of the X-ray CT apparatus 100 is various types of information for controlling the X-ray CT apparatus 100 to scan the patient P2, as described above. Specifically, the control information includes a position of the rotary frame 117 in the frame apparatus 110, a detection data acquisition state in the DAS 116, a position of the top board 133 in the bed apparatus 130, a reconstruction state of a CT image, and the like. Vital information of patients that are learning targets may be associated as labels with images instead of or in addition to the control information of the X-ray CT apparatus 100.

In this case, the workflow control function 155 determines whether the patient P2 has closed the door by additionally inputting current control information of the X-ray CT apparatus 100 and current vital information of the patient P2 to the opening/closing determination model MDL1 in addition to the image of the camera 200. The current vital information of the patient P2 may be acquired from, for example, a vital metering instrument that is not illustrated, such as an electrocardiogram and pulse oximeter, a sphygmomanometer, or a thermometer. The vital metering instrument such as an electrocardiogram and pulse oximeter, a sphygmomanometer, or a thermometer is another example of the “sensor.”

When the workflow control function 155 determines that the patient P2 has not closed the door on the basis of a confirmation result of the medical personnel member P1 and/or an output result of the opening/closing determination model MDL1, the workflow control function 155 returns to the process of 5102 and continues to induce the patient P2 to close the door of the CT room. Information representing a confirmation result of the medical personnel member P1 or information representing an output result of the opening/closing determination model MDL1 is an example of “second information.”

On the other hand, when the workflow control function 155 determines that the patient P2 has closed the door on the basis of a confirmation result of the medical personnel member P1 and/or an output result of the opening/closing determination model MDL1, the workflow control function 155 induces the patient P2 to lie down (to lie) on the top board 133 of the bed apparatus 130 as the next step of the scan workflow (step S108). For example, the workflow control function 155 may induce the patient P2 to lie down on the top board 133 of the bed apparatus 130 using the display 142 or the speaker 145.

In this manner, the workflow control function 155 permits transition to the next step S108 of the scan workflow and executes the process of the step S108 when two conditions that (i) the patient P2 self-reports closing of the door of the CT room and (ii) the medical personnel member P1 remotely confirms closing of the door of the CT room or the door of the CT room is determined to be closed using artificial intelligence are satisfied in the step S102.

Next, the workflow control function 155 determines whether the patient P2 has reported that he/she is lying down on the bed apparatus 130 (step S110).

For example, it is assumed that the patient P2 is induced to operate the first button 143a of the input interface 143 after lying down on the bed apparatus 130 and the first button 143a of the input interface 143 has been operated by the patient P2 as a result. In this case, the input interface 143 outputs a signal representing that the first button 143a has been operated to the processing circuit 150.

When the signal representing that the first button 143a has been operated cannot be acquired from the input interface 143, the workflow control function 155 determines that the patient P2 has not reported lying down on the bed apparatus 130. In this case, the workflow control function 155 returns to the process of S108 and continues to induce the patient P2 to lie down on the bed apparatus 130.

On the other hand, when the signal representing that the first button 143a has been operated is acquired from the input interface 143, the workflow control function 155 determines that the patient P2 has reported lying down on the bed apparatus 130. In this case, the workflow control function 155 determines whether the remotely located medical personnel member P1 has confirmed that the patient P2 is lying down on the bed apparatus 130 (step S112).

For example, when the medical personnel member P1 can confirm that the patient P2 is lying down on the bed apparatus 130 by viewing the image of the CT room displayed on the display 13, the medical personnel member P1 inputs a confirmation result representing that the patient P2 is lying down on the bed apparatus 130 to the input interface 12 of the terminal device 10. In other words, when the medical personnel member P1 can confirm that the patient P2 is lying down on the bed apparatus 130, the medical personnel member P1 inputs information for permitting transition to the next step of the scan workflow to the input interface 12 of the terminal device 10. Upon receiving this, the transmission control function 23 of the terminal device 10 transmits information indicating the confirmation result representing that the patient P2 is lying down on the bed apparatus 130 (permission for transition to the next step) to the X-ray CT apparatus 100 through the communication interface 11. When the communication interface 144 receives the information representing the confirmation result from the terminal device 10, the workflow control function 155 determines that the remotely located medical personnel member P1 has confirmed that the patient P2 is lying down on the bed apparatus 130.

The workflow control function 155 may determine whether the patient P2 is lying down on the bed apparatus 130 using artificial intelligence as the process of S112. For example, the workflow control function 155 determines whether the patient P2 is lying down on the bed apparatus 130 by inputting the image (i.e., the image of the inside of the CT room) of the camera 200 to a machine learning model (hereinafter, a lying determination model) MDL2 trained in advance to determine whether the patient P2 is lying down on the bed apparatus 130. The lying determination model MDL2 is another example of the “trained model.”

The lying determination model MDL2 may be, for example, a model implemented by a neural network such as a CNN like the opening/closing determination model MDLL The lying determination model MDL2 is a model supervised-trained on the basis of training data in which correct answer information representing whether patients that are learning targets lie down on the bed apparatus 130 is associated as labels with images of the inside of the CT room. This correct answer information may be, for example, a two-dimensional vector having a probability α3 representing that a patient that is a learning target is lying down on the bed apparatus 130 and a probability α4 representing that the patient that is a learning target does not lie down on the bed apparatus 130 as elements. The training data may be replaced with data sets obtained by combining input data and output data when images of the inside of the CT room are the input data and the correct answer information representing whether patients that are learning targets lie down on the bed apparatus 130 is the output data. Through training of the lying determination model MDL2 using such training data, the lying determination model MDL2 outputs information representing whether the patient is lying down on the bed apparatus 130 installed in the CT room when the image of the inside of the CT room is input.

For example, the workflow control function 155 may determine that the patient P2 does not lie down on the bed apparatus 130 when the lying determination model MDL2 outputs a vector in which the probability α4 is higher than the probability α3 (α4>α3) and determine that the patient P2 is lying down on the bed apparatus 130 when the lying determination model MDL2 outputs a vector in which the probability α3 is higher than the probability α4 (α3>α4).

The training data for learning the lying determination model MDL2 may be data sets in which correct answer information representing whether patients that are learning targets lie down on the bed apparatus 130 and control information of the X-ray CT apparatus 100 are associated as labels with images of the inside of the CT room. Vital information of patients that are learning targets may be associated as labels with images instead of or in addition to the control information of the X-ray CT apparatus 100.

In this case, the workflow control function 155 determines whether the patient P2 is lying down on the bed apparatus 130 by additionally inputting current control information of the X-ray CT apparatus 100 and current vital information of the patient P2 to the lying determination model MDL2 in addition to the image of the camera 200.

When the workflow control function 155 determines that the patient P2 does not lie down on the bed apparatus 130 on the basis of a confirmation result of the medical personnel member P1 and/or an output result of the lying determination model MDL2, the workflow control function 155 returns to the process of 5108 and continues to induce the patient P2 to lie down on the bed apparatus 130. Information representing an output result of the lying determination model MDL2 is another example of the “second information.”

On the other hand, when the workflow control function 155 determines that the patient P2 is lying down on the bed apparatus 130 on the basis of a confirmation result of the medical personnel member P1 and/or an output result of the lying determination model MDL2, the workflow control function 155 moves the top board 133 of the bed apparatus 130 to the inside of the rotary frame 117 (inside of the opening 160) as the next step of the scan workflow (step S114).

In this manner, the workflow control function 155 permits transition to the next step S114 of the scan workflow and executes the process of the step S114 when two conditions that (i) the patient P2 self-reports lying down on the bed apparatus 130 and (ii) the medical personnel member P1 remotely confirms that the patient P2 is lying down on the bed apparatus 130 or it is determined that the patient P2 is lying down on the bed apparatus 130 using artificial intelligence are satisfied in the step S108.

Next, the workflow control function 155 notifies a posture and an action (an action of temporarily holding the breath, or the like) that need to be taken by the patient

P2 in the frame apparatus 110 and a scanning part using the display 142 or the speaker 145 (step S116).

Next, the workflow control function 155 moves the display 142 in accordance with the posture of the patient P2 (step S118). For example, the workflow control function 155 moves the screen of the display 142 to the line of sight of the patient P2 by moving the robot arm 142a according to the posture of the patient P2.

Next, the workflow control function 155 induces the patient P2 to operate the input interface 143 when the patient P2 takes the posture and the action requested in the process of S116 and preparation for scanning is finished, using the display 142 or the speaker 145 (step S120).

Next, the workflow control function 155 determines whether the patient P2 has self-reported finishing of preparation for scanning (step S122). For example, when the first button 143a has been operated by the patient P2, the input interface 143 outputs a signal representing that the first button 143a has been operated to the processing circuit 150.

When the signal representing that the first button 143a has been operated cannot be acquired from the input interface 143, the workflow control function 155 determines that the patient P2 has not reported finishing of preparation for scanning. In this case, the workflow control function 155 returns to the process of S120 and continues to induce the patient P2 to operate the input interface 143 when preparation for scanning is finished.

On the other hand, when the signal representing that the first button 143a has been operated is acquired from the input interface 143, the workflow control function 155 determines that the patient P2 has reported finishing of preparation for scanning. In this case, the workflow control function 155 determines whether the remotely located medical personnel member P1 has confirmed finishing of preparation for scanning of the patient P2 (step S124).

For example, it is assumed that the medical personnel member P1 can confirm that the patient P2 takes the posture or the action requested in the process of S116 by viewing the image of the CT room and vital information of the patient P2 displayed on the display 13. In this case, the medical personnel member P1 inputs a confirmation result representing finishing of preparation for scanning of the patient P2 to the input interface 12 of the terminal device 10. In other words, when the medical personnel member P1 can confirm that the patient P2 takes the posture or the action requested in the process of S116, the medical personnel member P1 inputs information for permitting transition to the next step of the scan workflow to the input interface 12 of the terminal device 10. Upon receiving this, the transmission control function 23 of the terminal device 10 transmits information indicating the confirmation result representing finishing of preparation for scanning of the patient P2 (permission for transition to the next step) to the X-ray CT apparatus 100 through the communication interface 11. When the communication interface 144 receives the information representing the confirmation result from the terminal device 10, the workflow control function 155 determines that the remotely located medical personnel member P1 has confirmed finishing of preparation for scanning of the patient P2.

The workflow control function 155 may determine whether preparation for scanning of the patient P2 is finished using artificial intelligence as the process of S124. For example, the workflow control function 155 determines the posture of the patient P2 by inputting the image (i.e., the image of the inside of the CT room) of the camera 200 to a machine learning model (hereinafter, a posture determination model) MDL3 trained in advance to determine the posture of the patient P2 and determines whether preparation for scanning of the patient P2 is finished according to whether the determined posture of the patient P2 is the same as the posture requested in the process of S116. The posture determination model MDL3 is another example of the “trained model.”

The posture determination model MDL3 may be a model implemented by a neural network such as a CNN like the opening/closing determination model MDL1 and the lying determination model MDL2, for example. The posture determination model MDL3 is a model supervised-trained on the basis of training data in which correct answer information representing postures of patients that are learning targets is associated as labels with images of the inside of the CT room in which the patients that are learning targets lie down on the bed apparatus 130. This correct answer information may be, for example, a multi-dimensional vector having probabilities representing a plurality of postures that can be taken by patients as elements. Specifically, when there are three types of postures that can be taken by patients, lying face up, lying face down, and lying on one's side, the correct answer information is a three-dimensional vector having a probability representing lying face up, a probability representing lying face down, and a probability representing lying on one's side as elements. The training data may be replaced with data sets obtained by combining input data and output data when images of the inside of the CT room in which patients that are learning targets lie down on the bed apparatus 130 are the input data and correct answer information representing postures of the patients that are learning targets is the output data. Through training of the posture determination model MDL3 using such training data, the posture determination model MDL3 outputs information representing the posture of the patient P2 when an image of the inside of the CT room in which the patient P2 is lying down on the bed apparatus 130 is input.

For example, the workflow control function 155 determines that the patient P2 lying down on the bed apparatus 130 takes a posture of lying face up when the posture determination model MDL3 outputs a vector in which the probability representing lying face up is highest. Then, the workflow control function 155 determines that preparation for scanning of the patient P2 is finished if the posture requested in the process of S116 is the posture of lying face up and it is determined that the patient P2 is performing the action requested in the process of S116 from vital information of the patient P2 and determines that preparation for scanning of the patient P2 is not finished if not.

The training data for learning the posture determination model MDL3 may be data sets in which correct answer information representing postures of patients that are learning targets and control information of the X-ray CT apparatus 100 are associated as labels with images of the inside of the CT room in which the patients that are learning targets lie down on the bed apparatus 130. Vital information may be associated as labels with images instead of or in addition to the control information of the X-ray CT apparatus 100.

In this case, the workflow control function 155 determines the posture and the action of the patient P2 by additionally inputting current control information of the X-ray CT apparatus 100 and current vital information of the patient P2 to the posture determination model MDL3 in addition to the image of the camera 200.

When the workflow control function 155 determines that preparation for scanning of the patient P2 is not finished on the basis of a confirmation result of the medical personnel member P1 and/or an output result of the posture determination model MDL3, the workflow control function 155 returns to the process of S116 and induces the patient P2 to operates the input interface 143 when preparation for scanning is finished while notifying a posture and an action that need to be taken by the patient P2, a scanning part, and the like. The output result of the posture determination model MDL3 is another example of the “second information.”

On the other hand, when the workflow control function 155 determines that preparation for scanning of the patient P2 is finished on the basis of a confirmation result of the medical personnel member P1 and/or an output result of the posture determination model MDL3, the workflow control function 155 permits scanning to be executed as the next step of the scan workflow (step S126).

In this manner, the workflow control function 155 permits transition to the next step S126 of the scan workflow and executes the process of the step S126 when two conditions that (i) the patient P2 self-reports finishing of preparation for scanning and (ii) the medical personnel member P1 remotely confirms finishing of preparation for scanning of the patient P2 or it is determined that preparation for scanning of the patient P2 is finished using artificial intelligence are satisfied in the step S120.

The control device 118, the pre-processing function 152, the reconstruction processing function 153, and the image processing function 154 perform various processes for scanning when the workflow control function 155 permits execution of scanning. Specifically, the control device 118 performs main scan imaging or scan imaging while rotating the rotary frame 117 or tilting the frame apparatus 110. When the DAS 116 acquires detection data through main scan imaging or scan imaging, the pre-processing function 152 performs pre-processing on the detection data and generates projection data. The reconstruction processing function 153 performs reconstruction processing on the projection data generated by the pre-processing function 152 to generate a CT image. The image processing function 154 converts the CT image generated by the reconstruction processing function 153 into a three-dimensional image and a cross-section image data. Then, any function of the processing circuit 150 transmits the three-dimensional image and the cross-section image data of the CT image to the terminal device 10 through the communication interface 144 or causes the display 142 to display them.

Next, the workflow control function 155 determines whether to continue scanning on the basis of the scan workflow (step S128). For example, when scan imaging has been performed in the process of S126, the workflow control function 155 determines that scanning will continue because main scan imaging follows scan imaging. There are cases in which the same part is imaged many times or a plurality of parts are imaged even when main scan imaging is performed in the process of S126. Accordingly, the workflow control function 155 may determine that scanning will continue when the patient P2 is imaged many times through main scan imaging according to a scan workflow planned in advance.

When it is determined that scanning will continue, the workflow control function 155 returns to the process of S116, newly notifies a posture and an action that need to be taken by the patient P2 in the next scanning and a scanning part, and additionally moves the display 142 in accordance with the posture of the patient.

On the other hand, when it is determined that scanning will not continue, the workflow control function 155 moves the top board 133 of the bed apparatus 130 to the outside of the rotary frame 117 (outside of the opening 160) (step S130). Accordingly, processing of this flowchart ends.

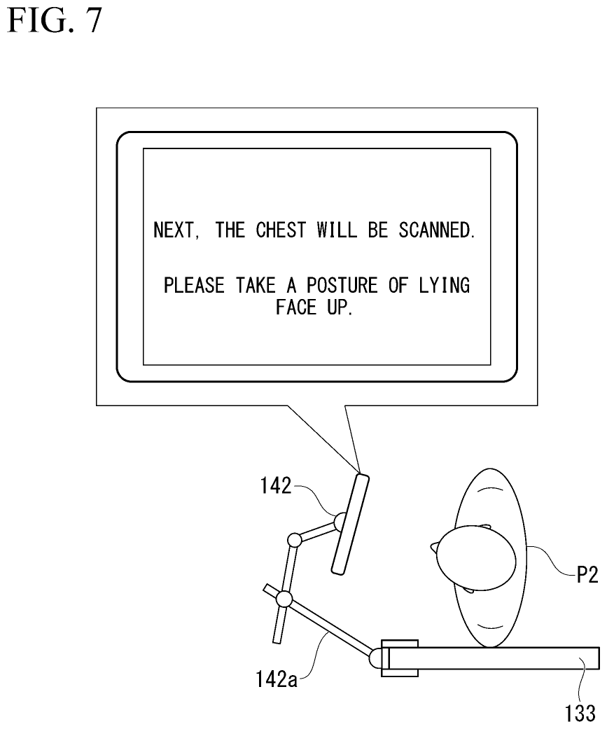

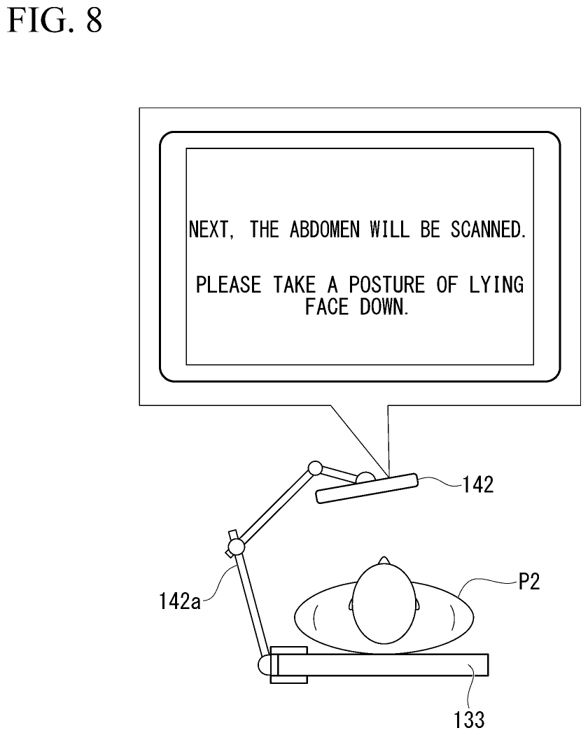

FIG. 7 and FIG. 8 are diagram schematically showing states in which the display 142 is moved in accordance with postures of the patient P2. When the patient P2 lies on the top board 133 in a posture of lying on his/her side, for example, as shown in FIG. 7, if the next scanning part is “chest,” the workflow control function 155 causes the display 142 to display that the next scanning part is “chest” and a posture that needs to be taken by the patient P2 to scan the “chest” is “lying face up.” Here, the workflow control function 155 moves the robot arm 142a according to change of postures of the patient P2 from “lying on his/her side” to “lying face up” to move the screen of the display 142 to the line of sight (in front of the face) of the patient P2 taking the posture of “lying face up,” as shown in FIG. 8.

It is assumed that the patient P2 lies face up according to an instruction displayed on the display 142 and scanning of “chest” is scheduled after execution of scanning. In this case, the workflow control function 155 causes the display 142 to display that a posture that needs to be taken by the patient P2 in the next scanning is “lying face down” and a part that will be scanned in that posture is “abdomen.” In this manner, the patient P2 can successively change postures on the top board 133 while understanding the next posture to be taken by him/her and the next part to be scanned.

### [Emergency Stop Flow of X-Ray CT Apparatus]

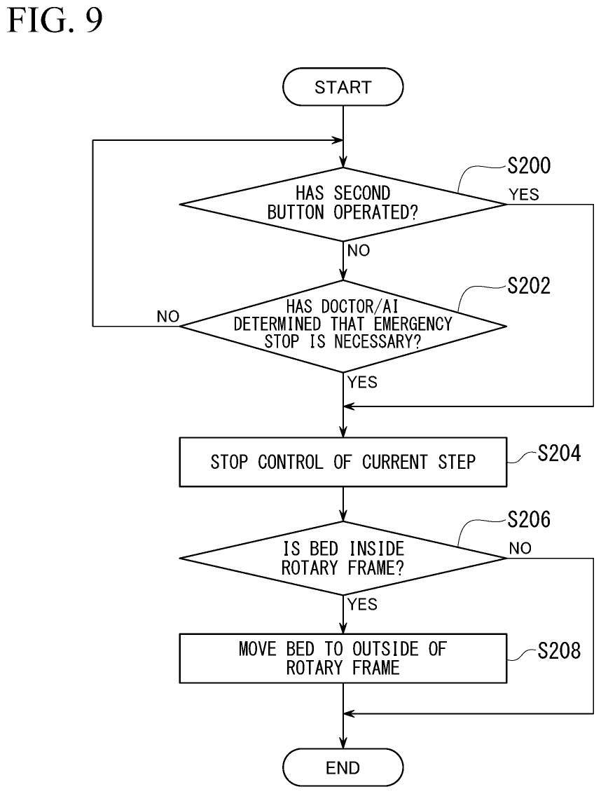

Hereinafter, a series of flowcharts for emergently stopping the X-ray CT apparatus 100 in an embodiment will be described. FIG. 9 is a flowchart showing a flow of a series of processes at the time of emergency stop of the X-ray CT apparatus 100 in an embodiment.

First, the workflow control function 155 determines whether the patient P2 has operated the second button 143b of the input interface 143 in order to emergently stop the X-ray CT apparatus 100 (step S200). Operation of the second button 143b is an example of a “predetermined instruction.”

To curb a misoperation such as erroneous pressing, for example, the workflow control function 155 may determine that the patient P2 has operated the second button 143b for the purpose of emergency stop when the second button 143b has been operated a predetermined number of times or more and may determine that the patient P2 has operated the second button 143b for the purpose of emergency stop when the second button 143b has been continuously operated for a predetermined time or longer. The workflow control function 155 may determine that the patient P2 has operated the second button 143b for the purpose of emergency stop when the second button 143b and the first button 143a have been simultaneously operated.

When the patient P2 does not operate the second button 143b of the input interface 143 for the purpose of emergency stop, the workflow control function 155 additionally determines whether the remotely located medical personnel member P1 has determined that emergency stop of the X-ray CT apparatus 100 is necessary (step S202).

For example, it is assumed that the medical personnel member P1 determines that the symptom of a side effect, such as vomiting or spasm, appears in the patient P2 and thus emergency stop is necessary by viewing an image of the CT room displayed on the display 13. In this case, the medical personnel member P1 inputs a determination result representing that emergency stop is necessary to the input interface 12 of the terminal device 10. Upon receiving this, the transmission control function 23 of the terminal device 10 transmits information indicating the determination result representing that emergency stop is necessary to the X-ray CT apparatus 100 through the communication interface 11. The workflow control function 155 determines that the remotely located medical personnel member P1 has determined that emergency stop of the X-ray CT apparatus 100 is necessary when the communication interface 144 receives the information indicating the determination result from the terminal device 10. The symptom of a side effect is an example of a “predetermined state.”

The workflow control function 155 may determine whether emergency stop of the X-ray CT apparatus 100 is necessary using artificial intelligence as the process of S202. For example, the workflow control function 155 determines whether emergency stop of the X-ray CT apparatus 100 is necessary by inputting an image (i.e., an image of the inside of the CT room) of the camera 200 to a machine learning model (hereinafter, an emergency stop determination model) MDL4 trained in advance to determine the necessity of emergency stop of the X-ray CT apparatus 100.

The emergency stop determination model MDL4 may be a model implemented by a neural network such as a CNN like the opening/closing determination model MDL1, the lying determination model MDL2, and the posture determination model MDL3, for example. The emergency stop determination model MDL4 is a model supervised-trained on the basis of training data in which correct answer information representing symptoms (particularly, symptoms with respect to side effects of CT examination) of patients that are learning targets is associated as labels with images of the inside of the CT room in which the patient that is a learning target is lying down on the bed apparatus 130. This correct answer information may be, for example, a multi-dimensional vector having probabilities representing a plurality of symptoms (which may also include normal states) that patients can get as elements. The training data may be replaced with data sets obtained by combining input data and output data when images of the inside of the CT room in which patients that are learning targets lie down on the bed apparatus 130 are the input data and correct answer information representing symptoms of the patients that are learning targets is the output data. Through training of the emergency stop determination model MDL4 using such training data, the emergency stop determination model MDL4 outputs information representing a symptom of the patient P2 when an image of the inside of the CT room in which the patient P2 is lying down on the bed apparatus 130 is input.

The training data for learning the emergency stop determination model MDL4 may be data sets in which correct answer information representing symptoms of patients that are learning targets and vital information of the patients that are learning targets are associated as labels with images of the inside of the CT room in which the patients that are learning targets lie down on the bed apparatus 130.

In this case, the workflow control function 155 determines a symptom of the patient P2 by additionally inputting current vital information of the patient P2 to the emergency stop determination model MDL4 in addition to the image of the camera 200.

When the workflow control function 155 determines that emergency stop of the X-ray CT apparatus 100 is necessary on the basis of a determination result of the medical personnel member P1 and/or an output result of the emergency stop determination model MDL4, the workflow control function 155 stops control (processing) of the current step of the scan workflow (step S204). For example, the workflow control function 155 stops execution of scanning upon determining that emergency stop of the X-ray CT apparatus 100 is necessary during processing of executing scanning in step S126.

In this manner, the workflow control function 155 stops control of the current step of the scan workflow when at least one of conditions that (i) the patient P2 requests emergency stop by operating the second button 143b of the input interface 143 and (ii) the medical personnel member P1 remotely determines that emergency stop is necessary or it is determined that emergency stop is necessary using artificial intelligence is satisfied.

Next, the workflow control function 155 determines whether the top board 133 of the bed apparatus 130 is present inside the rotary frame 117 (inside the opening 160) (step S206) and moves the top board 133 to the outside of the rotary frame 117 (outside of the opening 160) if the top board 133 is present inside the rotary frame 117 (step S208). Accordingly, processing of this flowchart ends.

According to the above-described embodiment, the X-ray CT apparatus 100 (an example of a medical image capturing apparatus) of a medical image diagnostic system 1 includes the processing circuit 150 that controls transition between a plurality of steps included in a scan workflow for scanning the patient P2 that is a subject. In a certain target step among the plurality of steps included in the scan workflow, the processing circuit 150 acquires a signal (an example of the first information) representing that the first button 143a has been operated from the input interface 143 when the patient P2 has operated the first button 143a of the input interface 143 in order to report his/her preparation state. Further, the processing circuit 150 acquires a confirmation result (an example of the second information) of the medical personnel member P1 from the terminal device 10 when the medical personnel member P1 has remotely confirmed the preparation state of the patient P2 using the terminal device 10 or acquires a determination result (another example of the second information) according to artificial intelligence when the preparation state of the patient P2 has been determined by the artificial intelligence in the target step. Then, the processing circuit 150 determines whether both conditions that (i) the patient P2 self-reports finishing of preparation for examination and (ii) the remotely located medical personnel member P1 confirms finishing of preparation of the patient P2 (or finishing of preparation of the patient P2 is determined by artificial intelligence) are satisfied, and controls transition to the next step of the scan workflow when the two conditions of (i) and (ii) are satisfied. Accordingly, it is possible to examine the patient P2 with safety and without impairing convenience even when the medical personnel member P1 such as a doctor or an engineer is not present near the X-ray CT apparatus 100.

### Modified Examples of Embodiment

Hereinafter, modified examples of the embodiment will be described. Although the workflow control function 155 moves the screen of the display 142 to the line of sight of the patient P2 by moving the robot arm 142a in the above-described embodiment, the present invention is not limited thereto. For example, the workflow control function 155 may control the projector 190 instead of controlling the robot arm 142a.

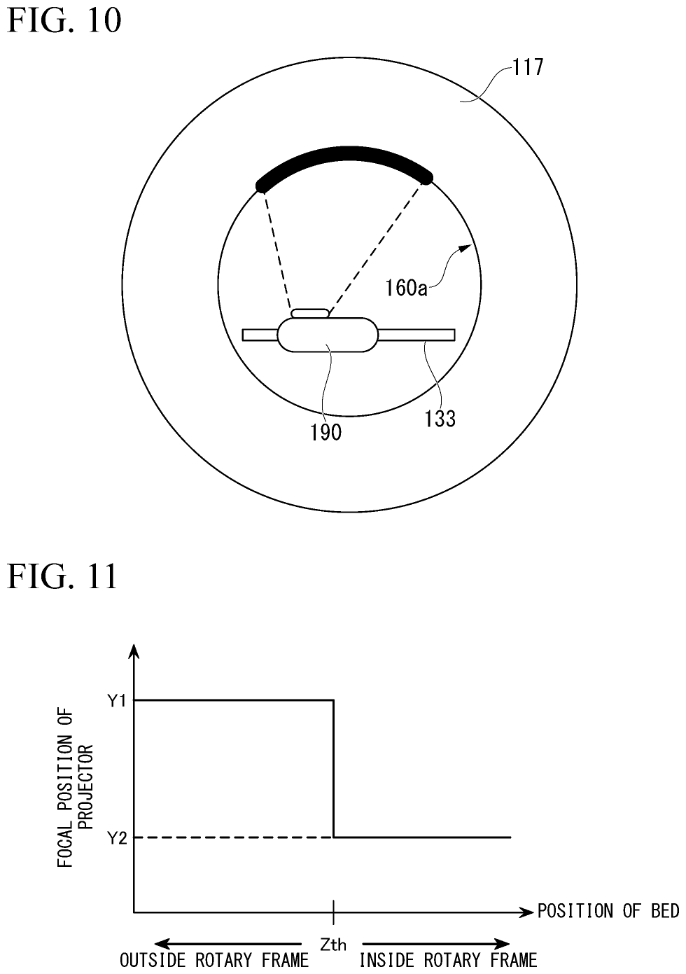

FIG. 10 is a diagram showing an example of the projector 190 in an embodiment. For example, the projector 190 may be attached to the top board 133 and the like. For example, the workflow control function 155 adjusts a focal position (projection position) of an image from the projector 190 to any of the wall surface 160a of the opening 160 of the frame apparatus 110 or the ceiling of the CT room depending on a relative position of the top board 133 with respect to the frame apparatus 110.

FIG. 11 is a diagram showing a focal position adjustment method. For example, it is assumed that the boundary between the outside and the inside of the rotary frame 117 (opening 160) is Zth, the position of the ceiling of the CT room is Yl, and the position of the wall surface 160a of the opening 160 of the frame apparatus 110 is Y2. In this case, the workflow control function 155 adjusts the focal position of the projector 190 to Y1 when the position of the top board 133 is within the boundary Zth, that is, the top board 133 is located outside the rotary frame 117 (opening 160). On the other hand, the workflow control function 155 adjusts the focal position of the projector 190 to Y2 when the position of the top board 133 is beyond the boundary Zth, that is, the top board 133 is located inside the rotary frame 117 (opening 160). Accordingly, it is possible to appropriately inform the patient P2 lying on the top board 133 of a posture that needs to be taken during scanning and a scanning part.

Although the processing circuit 150 of the X-ray CT apparatus 100 includes the workflow control function 155 in the above-described embodiment, the present invention is not limited thereto. For example, the processing circuit 20 of the terminal device 10 that can be used by the medical personnel member P1 may include the workflow control function 155.

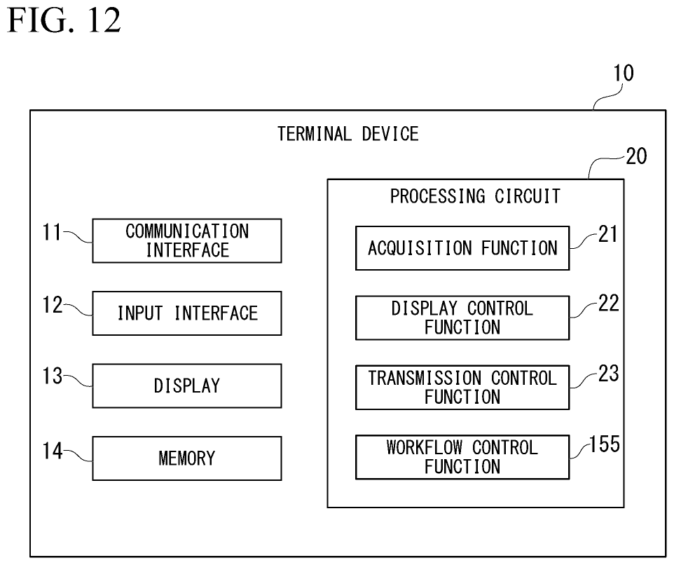

FIG. 12 is a diagram showing another configuration example of the terminal device 10 in an embodiment. As illustrated, the processing circuit 20 of the terminal device 10 further includes the workflow control function 155 included in the processing circuit 20 of the X-ray CT apparatus 100 in addition to the above-described acquisition function 21, display control function 22, and transmission control function 23.

For example, the workflow control function 155 of the terminal device 10 may determine whether both conditions that (i) the patient P2 self-reports finishing of preparation for examination and (ii) the remotely located medical personnel member P1 confirms finishing of preparation of the patient P2 (or finishing of preparation of the patient P2 is determined by artificial intelligence) are satisfied and control or permit transition to the next step of the scan workflow when the two conditions of (i) and (ii) are satisfied in steps S102, S108, and S120.

The workflow control function 155 may be included in the control device 118 of the frame apparatus 110 instead of the processing circuit 20 of the terminal device 10. That is, the control device 118 of the frame apparatus 110 may determine whether conditions that (i) the patient P2 self-reports finishing of preparation for examination and (ii) the remotely located medical personnel member P1 confirms finishing of preparation of the patient P2 (or finishing of preparation of the patient P2 is determined by artificial intelligence) are satisfied and control or permit transition to the next step of the scan workflow when the two conditions of (i) and (ii) are satisfied.

Although determining finishing of preparation of the patient P2 by a machine learning model implemented by a CNN or the like instead of confirming finishing of preparation of the patient P2 by the medical personnel member P1 is the condition (ii) for transition to the next step in the above-described embodiment, the present invention is not limited thereto.

For example, determining finishing of preparation of the patient P2 by a machine learning model (the aforementioned opening/closing determination model MDL1, lying determination model MDL2, or posture determination model MDL3) implemented by a CNN or the like instead of self-reporting finishing of preparation for examination by the patient P2 may be the condition (i) for transition to the next step. That is, the workflow control function 155 may determine whether conditions that (i) finishing of preparation of the patient P2 is determined by artificial intelligence and (ii) the remotely located medical personnel member P1 confirms finishing of preparation of the patient P2 are satisfied and control or permit transition to the next step of the scan workflow when the two conditions of (i) and (ii) are satisfied. In this manner, transition between steps of the scan workflow may be controlled without necessarily having a conversation with the patient P2. Information representing a determination result of artificial intelligence in this modified example is another example of the “first information.”

Although several embodiments have been described, these embodiments have been suggested as examples and are not intended to limit the scope of the invention. These embodiments can be implemented in other various forms and various omissions, substitutions and modifications are possible without departing from essential characteristics of the invention. These embodiments and modifications thereof are included in the scope and essential characteristics of the invention and also included in the invention disclosed in claims and the equivalents thereof.

What is claimed is:

1. A medical image diagnostic system comprising:
a processing circuit which is configured to control transition between a plurality of steps included in a workflow for examining a subject,
wherein the processing circuit is configured to acquire first information representing a preparation state of the subject in a first step among the plurality of steps, is configured to acquire second information representing permission for transition from the first step to a second step, and is configured to control transition from the first step to the second step on the basis of the first information and the second information.

2. The medical image diagnostic system according to claim 1, further comprising an input interface operable by the subject,
wherein the processing circuit is configured to acquire information input by the subject through the input interface in the first step as the first information.

3. The medical image diagnostic system according to claim 1, further comprising a sensor which is configured to detect a state of the subject,
wherein the processing circuit is configured to determine a preparation state of the subject on the basis of the state of the subject detected by the sensor in the first step and is configured to acquire a determination result of the preparation state of the subject as the first information.

4. The medical image diagnostic system according to claim 3, wherein the processing circuit is configured to input data representing the state of the subject detected by the sensor in the first step to a trained model and is configured to determine a preparation state of the subject on the basis of data output from the trained model, and
wherein the trained model is a model supervised-trained on the basis of training data in which correct answer output data representing preparation states of subjects that are learning targets is associated as labels with input data representing states of the subjects that are learning targets.

5. The medical image diagnostic system according to claim 1, further comprising a communication interface which is configured to communicate with an external terminal device through a network,
wherein the processing circuit is configured to acquire information received from the external terminal device through the communication interface in the first step as the second information.

6. The medical image diagnostic system according to claim 2, further comprising a sensor which is configured to detect a state of the subject,
wherein the processing circuit is configured to determine whether to permit transition from the first step to the second step on the basis of the state of the subject detected by the sensor in the first step and is configured to acquire a determination result of permission for the transition as the second information.

7. The medical image diagnostic system according to claim 6, wherein the processing circuit is configured to input data representing the state of the subject detected by the sensor in the first step to a trained model and is configured to determine whether to permit transition from the first step to the second step on the basis of data output from the trained model, and
wherein the trained model is a model supervised-trained on the basis of training data in which correct answer output data representing preparation states of subjects that are learning targets is associated as labels with input data representing states of the subjects that are learning targets.

8. The medical image diagnostic system according to claim 1, further comprising:
an input interlace operable by the subject; and
a sensor which is configured to detect a state of the subject,
wherein the processing circuit is configured to stop control of the first step when a predetermined instruction is input by the subject through the input interface in the first step or when the sensor detects that the subject is in a predetermined state in the first step.

9. A medical image diagnostic method, of a processing circuit, comprising:
controlling transition between a plurality of steps included in a workflow for examining a subject;
acquiring first information representing a preparation state of the subject in a first step among the plurality of steps;
acquiring second information representing permission for transition from the first step to a second step; and
controlling transition from the first step to the second step on the basis of the first information and the second information.

10. An input device connected to a frame of a medical image capturing apparatus for scanning a subject in a wired or wireless manner and operable by the subject during examination.

11. A display device which is configured to display an image for inducing a subject to take a posture or an action suitable for scanning performed by a medical image capturing apparatus.

12. The display device according to claim 11, further comprising:
a robot arm provided on a bed of the medical image capturing apparatus;
a display provided on the robot arm; and
a processing circuit which is configured to control the robot arm depending on a posture of the subject.

13. The display device according to claim 11, further comprising:
a projector which is configured to project a video; and
a processing circuit which is configured to control a position of a video projected by the projector to any of a ceiling of a room in which the medical image capturing apparatus is installed and the inside of a frame of the medical image capturing apparatus depending on a position of the bed of the medical image capturing apparatus.
